(19) United States
(12) Patent Application Publication
(10) Pub. No.: US 2019/0159412 A1
(43) Pub. Date: May 30, 2019

(54) IN-GROUND MOISTURE SENSOR AND SYSTEM

(71) Applicants: Jerome GUIDISH, Naples, FL (US); Douglas Guidish, Naples, FL (US)

(72) Inventors: Jerome GUIDISH, Naples, FL (US); Douglas Guidish, Naples, FL (US)

(21) Appl. No.: 16/204,283

(22) Filed: Nov. 29, 2018

Related U.S. Application Data
(60) Provisional application No. 62/592,179, filed on Nov. 29, 2017.

(51) Int. Cl. A01G 25/16; G01N 33/24; G01N 27/22

(52) U.S. Cl. CPC A01G 25/167; G01N 27/223; G01N 33/246

(57) ABSTRACT

A system for controlling watering is disclosed. The system uses an in-ground moisture sensor and an end user device. The system uses at least one in-ground probe which has a ground engagement portion and an upper portion. The upper portion houses a communication module and a power module. The system also uses an end user device that has a communication module and a water-shut off valve.

### BACKGROUND OF THE INVENTION

### 1. Field of the Invention

The field of the invention is a sensor for detecting level of moisture in ground and a system for using an array of such sensors.

### 2. Background of the Invention

In various embodiments, the invention provides a means for measuring moisture levels, communicating with sensors of moisture levels, and adjusting watering times for sprinkler systems in response to moisture levels. In another embodiment, the system comprises a stand-alone sensor that is placed in the ground and sends data directly to the end user. In yet another embodiment, the system uses multiple probes to send soil health data to an end user's device.

In one embodiment, the invention comprises a probe and a controller for the watering system which communicates with the probe. The controller is compatible with a pre-existing water dispersion system, such as a lawn sprinkler system. Each probe comprises a sensor and a communication module. In one version, at regular intervals, each probe sensor sends telemetry data to the communication module, which in turn communicates with the controller. The controller thereafter determines the time of the next watering event, as well as the duration of the watering event.

A common problem with moisture sensors is communication and durability. Many moisture sensors include only a short term power supply which is not rechargeable. Other sensors corrode after being installed in the ground.

A need exists in the art for a device that includes probes that use a rechargeable power source and do not readily corrode. Further, the probes should include reliable communication means.

### SUMMARY OF INVENTION

An object of the invention is to create a system of controlling watering. An advantage of the invention is that it retrofits existing sprinkler systems and the like to include a controller with multiple probes.

A further object is to provide a system which is compatible with existing watering deployments. A feature of the invention is that in one embodiment it comprises a probe and controller which ties into an existing system. An advantage of the invention is that it adds adaptive control to a legacy watering system.

Another object of the invention is to provide a system that prevents overwatering. A feature of the invention is that the combination of controller and probes ensure that only a needed amount of water is dispersed. An advantage of the invention is that it prevents unnecessary watering. Further, the probe also prevents under-watering which could kill plant life and damage the soil. The probe allows the use of an optimum amount of water for the variety of plant growing at a particular location.

A further object of the invention is to provide a system compatible with large-scale deployments. A feature of the invention is that multiple probes may be deployed on a large geographical area, such as a golf course. An advantage of the system is that it prevents overwatering.

Yet another object of the invention is to provide a sensor with a long lasting power supply. A feature of the invention is that each probe contains a solar panel on a top surface and a power storage device, such as a lithium-polymer battery or a capacitor. Further each probe is in a power-saving sleep mode except when communicating with the controller. An advantage of the system is that the probes may be deployed for long time periods.

A further object of the invention is to provide a probe which does not corrode. A feature of the invention is that the ground engagement moisture sensor uses materials which do not corrode readily. For the cathode and anode one embodiment uses stainless steel and graphite. In another embodiment, the probe circuitry is encased and physically isolated from the moisture, preventing any corrosion. This embodiment uses capacitance of measure moisture levels. An advantage of the system is that the probes do not corrode when deployed in the ground and can remain installed for many years.

Another object of the invention is to provide a system with controllers which communicate wirelessly. A feature of the invention is in one embodiment, each controller contains at least one wireless radio, such as 802.11 wireless local area network that is used to communicate with external services, and another radio to communicate with the probes. Another embodiment uses Long Range Radio signals (LoRA). A further embodiment utilizes cellular technology, specifically, LTE Cat M1 (also known as LTE-M). A further embodiment uses Narrowband IoT (NB-IoT), SigFox and LoraWan, or a combination of several options. A benefit of using cellular is the removal of the need for a central hub station, allowing our devices to communicate directly with the user. A benefit of the invention is that the controllers can obtain external data sources such as precipitation forecasts.

An additional object of the invention is to support providing feedback for the controller. A feature of the invention is that the controller obtains weather information from probes, such as changing moisture level and whether it is currently raining. A benefit of the system is that the controller can iteratively change the planned watering pattern based on expected precipitation as well as current measurements from sensors.

A further object of the invention is to support reporting of current weather events to the controller. A feature of the system is that each probe can provide feedback about instantaneous weather events through the use of sensors, including an accelerometer. A benefit of the system is that individual probes can detect precipitation as well as confirm that watering events are occurring as planned.

An additional object of the invention is to support long distance communication. A feature of the invention is that each probe includes long distance wireless interfaces such as LoRa, LoraWan, Nb-IoT, LTE Cat M1, SigFox and others. A benefit of the invention is that in some embodiments, the system can be deployed in very large geographical areas, such as multiple holes of a golf course, a university campus, or a field.

A further object of the invention is to provide a probe which is similar in composition to the ground. A feature of the invention is that each resistance-based probe uses components made from Aluminum Oxide ceramic foam. The alternative capacitance based sensor does not require the ceramic foam. A benefit of the invention is that the material is similar to ground and prevents reactions with the soil.

An in-ground moisture sensor system is described.

### BRIEF DESCRIPTION OF DRAWING

The invention together with the above and other objects and advantages will be best understood from the following detailed description of the preferred embodiment of the invention shown in the accompanying drawings, wherein:

FIG. 1 depicts an overview of a probe according to one embodiment of the invention;

FIG. 2 depicts an overview of a controller according to one embodiment of the invention;

FIG. 3 depicts a flow chart of the operation of the water controller in one embodiment of the invention;

FIG. 4 depicts a chart of energy output as measured in one embodiment of the invention;

FIG. 5 depicts a chart of solar radiation in tests of one embodiment of the invention; and

FIG. 6 depicts a chart of accelerated corrosion data per one embodiment of the invention.

### DETAILED DESCRIPTION OF THE INVENTION

The foregoing summary, as well as the following detailed description of certain embodiments of the present invention, will be better understood when read in conjunction with the appended drawings.

To the extent that the figures illustrate diagrams of the functional blocks of various embodiments, the functional blocks are not necessarily indicative of the division between hardware circuitry. Thus, for example, one or more of the functional blocks (e.g. processors or memories) may be implemented in a single piece of hardware (e.g. a general purpose signal processor or a block of random access memory, hard disk or the like). Similarly, the programs may be stand-alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

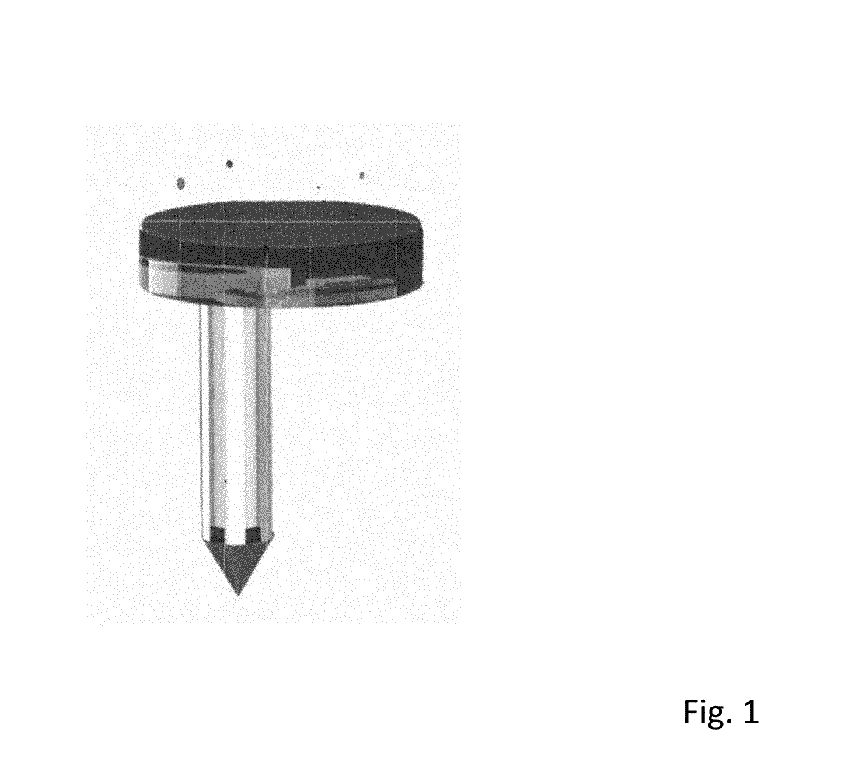

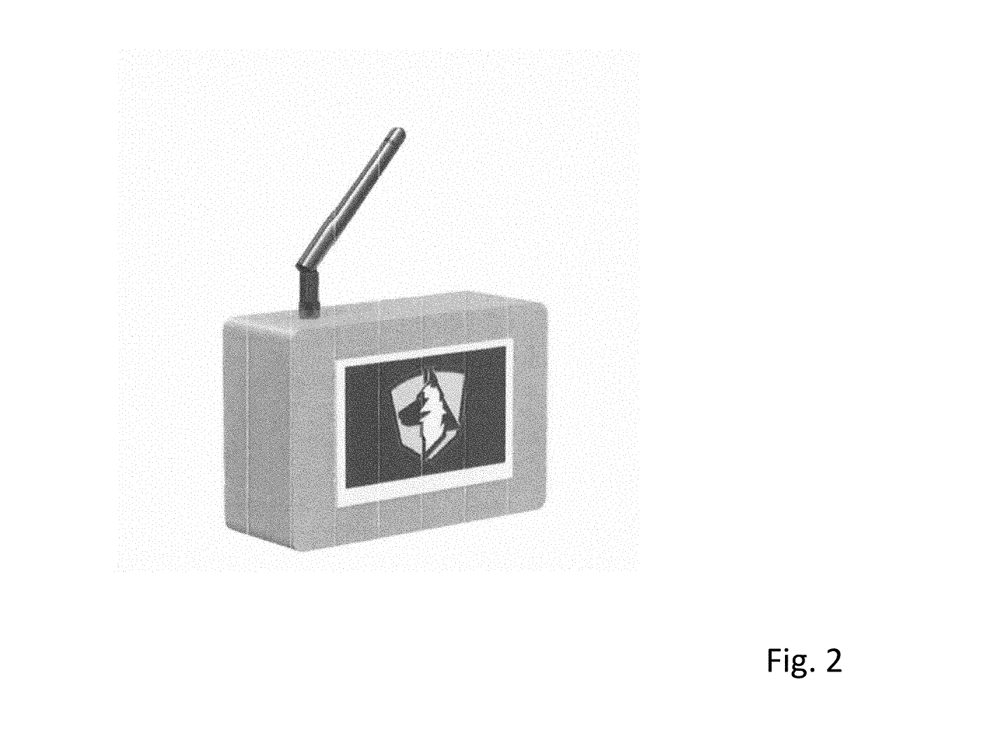

As shown in FIGS. 1 and 2, in one embodiment the main system comprises two components, namely the moisture probe of FIG. 1 and the main controller of FIG. 2. In another embodiment, each moisture probe communicates directly with an end-user device, such as a smart phone. In this embodiment, the central controller is simulated on the end user's phone and the data is sent directly to the end user device or computer. A dedicated component is used in embodiments which operate large scale irrigation systems. In one embodiment, the probe measures the moisture level in the soil. In another embodiment, the probe includes other sensors to measure soil health, such as pH level, nutrient level, oxygen level, Nitrogen-Phosphorous Potassium (NPK) levels, and other measurements.

Design methodology is directed towards exceeding specification within the constraints of discrete component limitations. The primary and most significant moisture probe constraint is the physical design package and the minimum required hardware to provide required core functionality of reliably and frequently reporting moisture data. As such, the moisture probe is optimized for the highest specification possible within physical constraints. The main controller has much less rigid specification. The main controller must have a wireless subsystem robust enough to serve the majority of residential environments, otherwise design is optimized towards compactness and aesthetic without compromise in ease of use and accessibility. In one embodiment, the controller is simulated on a general purpose computing device, such as a cellular phone. This embodiment allows the end user to avoid installing a physical controller, but instead can manage the probes and controlled systems (such as a water valve) using a general purpose device. In one such embodiment, the controller functions are handled by an app running on a cellular phone.

The electrical design of the system and the chemical reactions pertinent to the delivered design have been simulated to an extent that has established these values to an acceptable degree of confidence. From simulation results the applicant has come to the conclusion that the projected lifecycles for the discrete subcomponents far exceed system requirements; the deviation from theory present in real-use should be observable but negligible with regards to the required parameters. For example, the calculated power design can sustain the system for 44 days of darkness or an average of 2 hours of daylight, it is well within reason to assume that environmental conditions will exceed these minimum parameters. The specified lifecycle of 5 years can be met with a design featuring overwhelming excess to account for any incurred or unforeseen errors.

One of the design specifications is that the battery life of the probe will last a minimum of 5 years. The probe is active for only 60 seconds every hour, drawing 407 mW at 80 mA. Over a 24 hour period, this is equivalent to 81.4 mW per hour. When dormant, the probe draws 33 W. Over a 24 hour period while dormant the probe draws 787.6 W per hour. Therefore, over a day the total power drawn is approximately 81.19 mW. By dividing 81.19 mW by 24 hours we can determine the average power to be drawn every hour, which is approximately 3.38 mW per hour. To verify that the battery can supply sufficient current, we divide the power generated by the micro-controller by the voltage of the battery, which is approximately 939.7 A. This means that the average current drawn from the battery per hour is 0.94 mA,which is in the allowable powering range of the battery which is from 100 mA to 120 mA. In other embodiments, energy storage devices such as capacitors are used.

To determine the battery life of the battery divide the capacity of the battery by the current of the microcontroller times the battery efficiency which is 100 mAh divided by 939.7 A multiplied by 70%. This provided a theoretical value of approximately 74.5 hours or 3 days. The value of 100 mAh was chosen since the range allowable is between 100-120 mAh. To remain conservative we chose 100 mAh. It is important to note that these calculation were based off one battery. The probe could potentially house 4 batteries which would increase the battery life to 12 days without any recharging.

Each probe is powered by a 3.7 volt lithium ion battery. The solar panel runs on 6 volts and is compatible to charge the battery of 3.7 volts. The microcontroller runs on 3.3 volts. Data is collected for 5 seconds every 5 minutes. In a 24 hour day the probe will run for 24 minutes (0.4 hours) in active mode and 1,416 minutes (23.6 hours) in sleep mode. The microcontroller has a daily load of 106.38 milliwatts which provides an estimation load of 4.43 milliwatts per hour. The microcontroller will be pulling an approximate current of 1.20 milliamps per hour from the battery.

| Active Mode | Sleep Mode |
|---|---|
| Voltage - 3.3 V | Voltage - 3.3 V |
| Power - 264 mW | Power - 33 μW |
| Current - 80 mA | Current - 10 μA |

The power calculations are as follows:

$$Active Load \text{:} \frac{264 mW \times 24 min \times 1 hr}{60 min} = 105.6 \frac{mW}{hr}$$

$$Sleep Load \text{:} \frac{33 µW \times 1416 min \times 1 hr}{60 min} = 778.8 \frac{µW}{hr} = 0.7788 \frac{mW}{hr}$$

$$Daily Consumption \text{:} \frac{106.38 mW per day}{3.7 v ( Battery )} = 28.75 mA per day$$

$$Hourly Consumption \text{:} \frac{4.43 \frac{mW}{h}}{3.7 v ( Battery )} = 1.120 \frac{mA}{h}$$

The dispersion efficiency on the battery is 70%. The battery provides a capacity rate of 1800 milliamp hours or 2500 milliamp hours. The microcontroller only requires 1.120 milliamps per hour. Depending on the capacity of the battery the probe can last for approximately 44 days with a capacity rate of 1800 mAh, or 61 days with a capacity rate of 2500 mAh, before needing a complete full recharge.

The battery life calculations are as follows:

$${Battery}_{1800} \text{:} \frac{1800 mAh}{1.120 mA} \times 70 \% = 1051.75 hours \approx 44 days$$

$${Battery}_{2500} \text{:} \frac{2500 mAh}{1.120 mA} \times 70 \% = 1461.77 hours \approx 61 days$$

The solar panel operates at 6 volts and is capable of charging a 4.2 volt battery with an output of 100 milliamps per hour. The charge controller will regulate the voltage to provide the battery with an output current of 100 milliamps at 4 volts. The battery has a capacity rating of 1800 mAh or 2500 mAh, in various embodiments.

Since the initial capacity rate of the battery is very high compared to current drawn by the microcontroller, 1.120 mA per hour, it is reasonable to state that the battery will never have to be charged from complete discharge to full charge. Especially considering the solar panel has the capability to produce and output far greater than what the microcontroller draws per hour.

Now considering the solar panel's output is contingent to the amount of sunlight it receives, it's possible to determine the minimal output required to meet the daily load of the microcontroller. The daily load on the battery is approximately equivalent to 106.38 milliwatts. The shortest days in the year are in December, which records indicate that the shortest day is approximately 10 hours of sunlight, and if the skies that day are partially cloudy that results in only 5 hours of sunlight. Dividing 106.38 milliwatts by 5 is equivalent to 21.28 milliwatt, the solar panel needed to support the daily load of the microcontroller so that the battery is not depleted. If there are only 2 hours of sunlight then that value increases to 53.19 milliwatt panel and if there is only one hour of sunlight then the panel must be equal to 106:3 8 milliwatts to support the daily load.

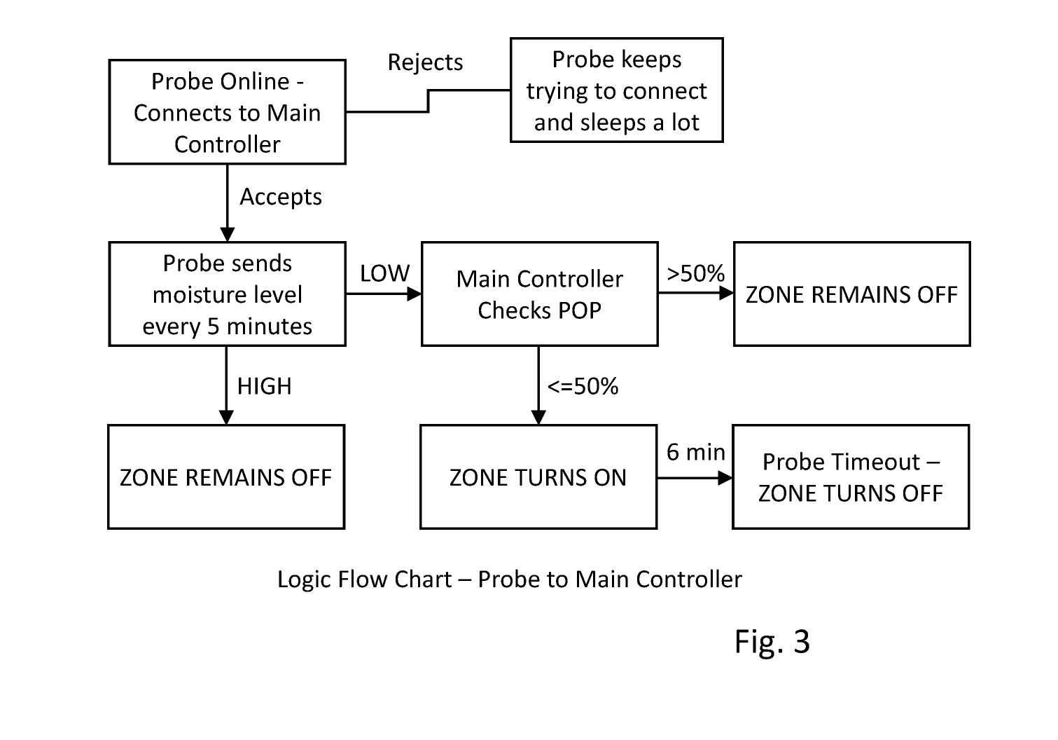

The system operation flowchart is presented in FIG. 3.

### Detailed Components

The following components are used in one embodiment of the controller shown in FIG. 2:

Housing: Printed plastic, UV stable molded ABS should be used for marketable product.

Microcontroller: GC-ESP8266 Launcher, ideally final model should feature modified or custom fabricated hardware

DC Solid State Relay: Alternative relay design with a smaller physical package is desirable

Touch screen: 5.0″ Nextion display is used, may be acceptable for final product, comparable choices should use JT AG or as few I/O pins as possible to simplify microcontroller specification.

The following components are used in one embodiment of the probe shown in FIG. 1:

Housing: a polyester resin, which provides provide a fully waterproof design.

Anode and Cathode: Carbon graphite and stainless steel respectively. Conductive planes and material choices may be altered for cost optimization provided that alternative materials exhibit comparable or superior chemical stability.

Solar panel: Rated 6V @ 100 mA, 100 mm diameter

Ceramic foam: Aluminum Oxide

Charge controller: Includes safety shut off and overcharge control

Battery: 1800/2500 mAh rechargeable battery or equivalent power source: including thermocouple for temperature safety and voltage safety, for embodiments using lithium based batteries.

Microcontroller: ESP8266-NodeMCU: includes voltage regulator and additional UOs.

Wheatstone bridge: Comparison of an unknown resistance in circuit.

Cathode interface threads: Stainless steel material to interface with stainless cathode.

Wiring: All wiring is soldered and a heatshrink is used to protect short circuits.

Hall Effect Switch: Provides the possibility to reset the microcontroller after it is encased inside of the polyester resin.

Many of the components that must be purchased include the electronic devices present in the system, such as the microcontroller, touch screen, charge controller, relays, and solar panel. Other parts of the system may be fabricated, such as the anode and cathode, the probe and main controller housings, and the ceramic foam. The longest lead times in the project are the result of component manufacture times, particularly when it comes to the stainless steel cathode and the machining of the aluminum oxide foam. However, manufacture times may be cut down significantly by casting these components rather than having them machined.

### Solar Panel Performance

The moisture probe is equipped with a 6 volt solar panel with the ability to output 100 milliamps. The voltage is regulated to 4 volts. The solar panel has a conversion efficiency of approximately 14.7%. The area of the solar panel is 0.008 meters squared. The energy output of the solar panel is equal to the multiplication of: area of solar panel, conversion efficiency, and solar radiation.

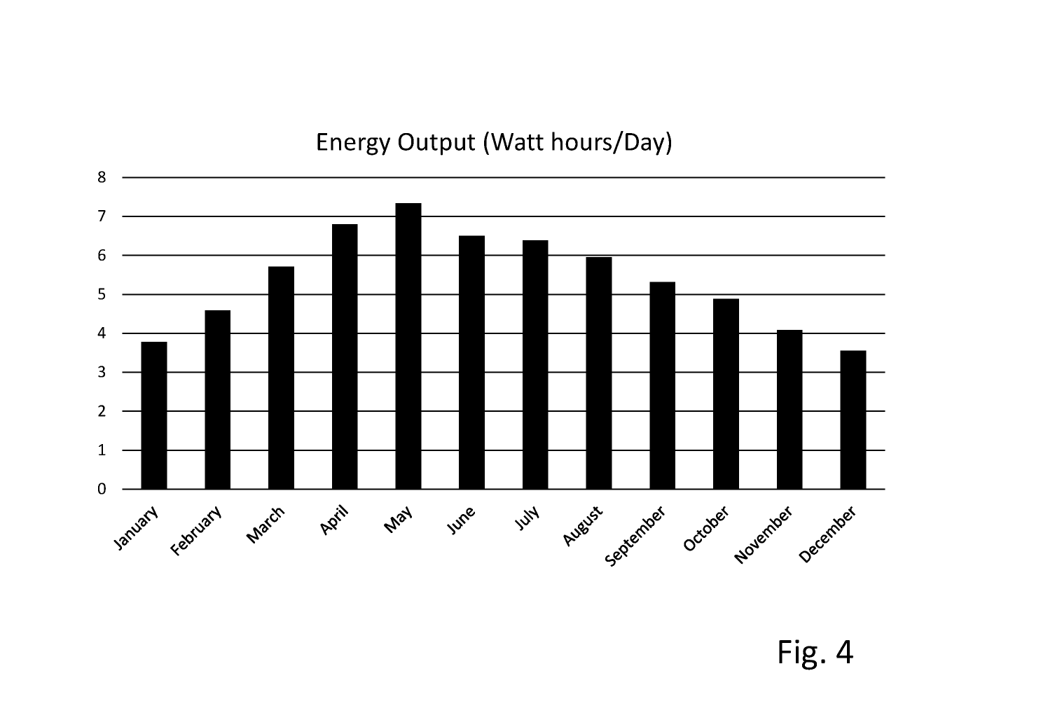

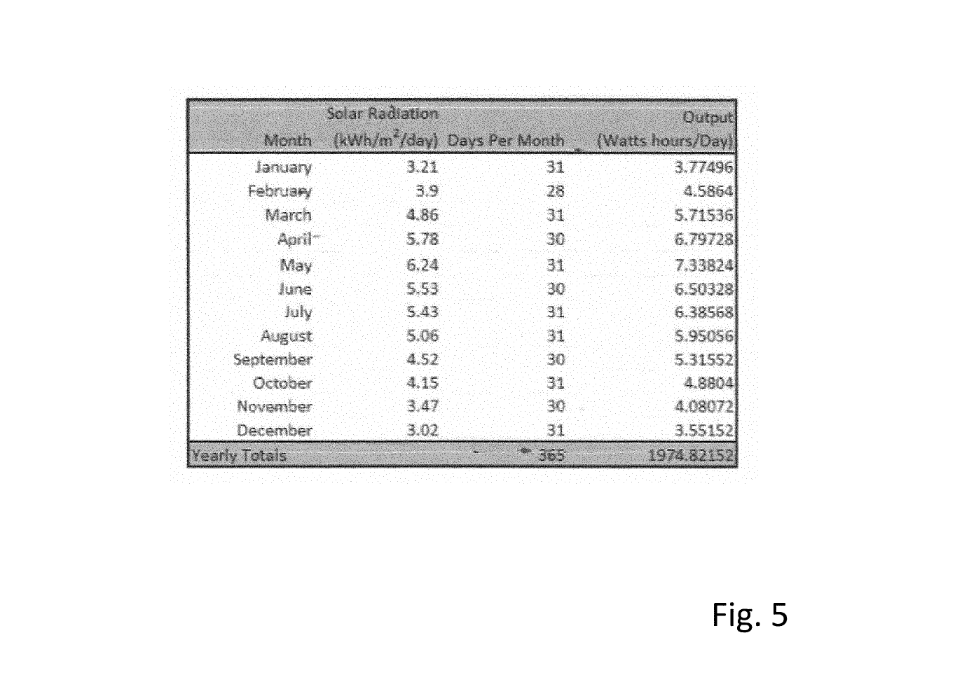

FIG. 4 depicts the daily output of the solar panel, based on the month of the year. FIG. 5 depicts the amount of solar radiation and the output from the solar panel.

As shown in FIGS. 4 and 5, the daily energy output for a day in December for the solar panel is 3.55 watt hours. Therefore, the hourly output would be approximately 148 milliwatt hours; 5 hours of sunlight would equal approximately 740 milliwatts. Considering that the average hourly output would be approximately 148 milliwatts this is more than enough to restore the depleted battery charge for the daily load of the microcontroller. The graph below shows the daily output of the solar panel based on the month of the year.

The power design results in a probe with a life span of 5 years.

### Oxygen Reduction Performance

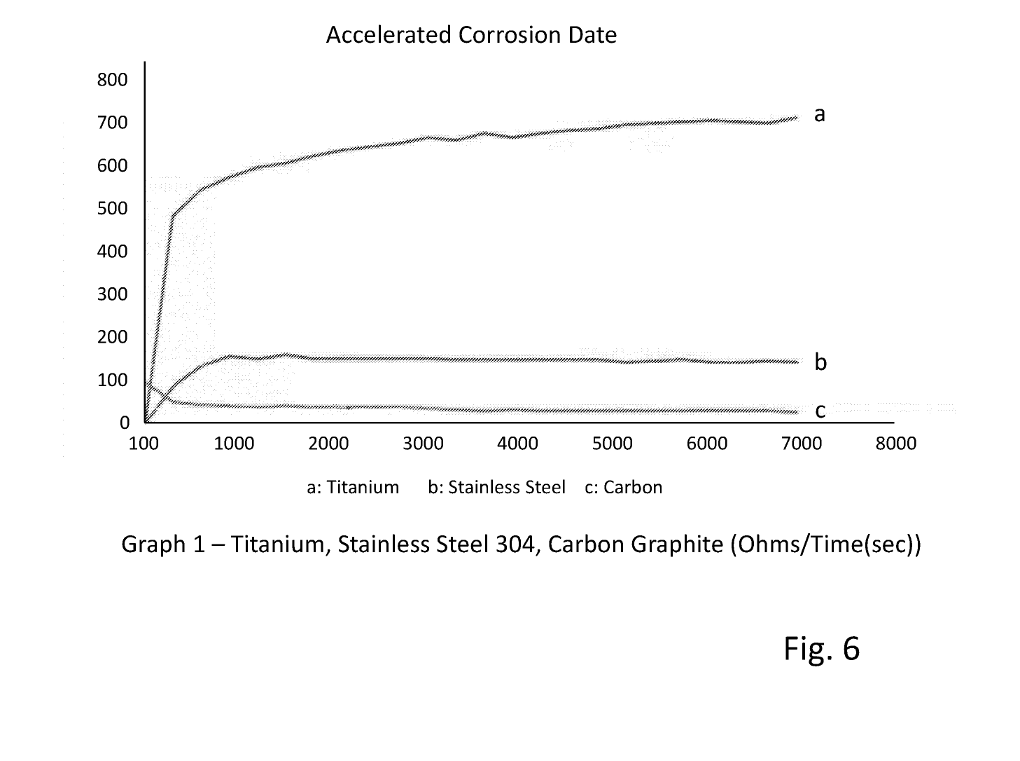

The corrosion data is shown in FIG. 6.

Oxygen Reduction cycle of the anode and cathode assembly were simulated with accelerated load testing far in excess of real-use load. For the testing the anodes were subject to 12 volts at 20 amps, substantially higher than the normal operating conditions which are 300 m V at 60 mA. Testing at operating conditions would induce a much lower oxidation of the anode. The collected data was used to compare three different materials as best candidates for use as the anode. Due to the electronegative nature of oxygen atoms, they are attracted to the anode, and therefore the anode has a high propensity for oxidation corrosion and material degradation. The graph shown in FIG. 6 provides a very clear graphical representation of resistance levels measured over time. The graph shows 7000 seconds of continuous operation at elevated voltage and current levels.

A microcontroller/datalogger was used to measure the resistance between the anode and cathode in a water bath on 5 minute intervals. Carbon Graphite was determined to have the best overall properties with the only negative being that the material can dissolve under the high load conditions given in the test. Additional testing was performed at the actual operating voltage and current, and no visible decay was observed over the same time period. The importance of having a steady resistance level over time is due to the value being so important with respect to the embedded control logic. The entire system relies on these data values being accurate over time, as well as a good working range between 10-300 ohms. The major concern with Stainless Steel is that the iron oxides forming on the exterior of the anode will clog of the pores of the aluminum oxide material and eventually cause a short circuit in the probe. The results derived indicate a very generous design factor over prescribed specification.

In one embodiment, the issue of corrosion is avoided by using a different technique to measure soil health. Rather than using a resistive device, the embodiment uses capacitance to measure the moisture levels. By using capacitance, the system can use circuitry which is physically isolated from the moisture and is therefore not susceptible to corrosion.

Although exemplary implementations of the invention have been depicted and described in detail herein, it will be apparent to those skilled in the relevant art that various modifications, additions, substitutions, and the like can be made without departing from the spirit of the invention and these are therefore considered to be within the scope of the invention as defined in the following claims.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions and types of materials described herein are intended to define the parameters of the invention, they are by no means limiting, but are instead exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the terms “comprising” and “wherein.” Moreover, in the following claims, the terms “first,” “second,” and “third,” are used merely as labels, and are not intended to impose numerical requirements on their objects. Further, the limitations of the following claims are not written in means-plus-function format and are not intended to be interpreted based on 35 U.S.C. § 112, sixth paragraph, unless and until such claim limitations expressly use the phrase “means for” followed by a statement of function void of further structure.

The embodiment of the invention in which an exclusive property or privilege is claimed is defined as follows:

1. An in-ground moisture sensor system comprising:
at least one probe having a ground engagement portion and an upper portion wherein said upper portion houses a communication module and a power module;
at least one end user device in communication with said probe using the probe's communication module and a water-shut off valve.

2. The system of claim 1 wherein said probe power module comprises a lithium-polymer battery and a solar cell embedded on the probe upper portion.

3. The system of claim 1 wherein the system further comprises a center controller that is in communication with the at least one probe and the at least one end user device.

4. The system of claim 3 wherein said probe communication module comprises a wireless communication system which forwards readings of the at least one probe to the center controller.

5. The system of claim 3, wherein the at least one end user device comprises the center controller, so that the end user device directly communicates with the at least one probe.

6. The system of claim 1 wherein said probe comprises an aluminum oxide ceramic foam and electrodes comprising stainless steel and graphite.

7. The system of claim 1 wherein the at least one probe comprises a probe for measuring moisture level.

8. The system of claim 7 wherein the at least one probe further comprises at least one of a probe for measuring pH level, a probe for measuring nutrient level, a probe for measuring oxygen level, and a probe for measuring Nitrogen-Phosphorous Potassium level.

9. The system of claim 7 wherein the probe measures moisture level by using capacitance, and the probe is sealed from moisture.

10. The system of claim 1 wherein said controller comprises a wireless interface to an external data source wherein said external data source provides a weather forecast.
